(19) United States
(12) Patent Application Publication
(10) Pub. No.: US 2004/0087795 A1
(43) Pub. Date: May 6, 2004

(54) Novel processes for the preparation of 4-phenylpiperidine derivatives

(76) Inventors: Gary Thomas Borrett, Hertfordshire (GB); Michael Fedouloff, Hertfordshire (GB); Mark Jason Hughes, Hertfordshire (GB); Andrew Colin Share, Hertfordshire (GB); John Bryce Strachan, Hertfordshire (GB); Peter Szeto, Hertfordshire (GB); Martyn Voyle, Hertfordshire (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/333,274

(22) PCT Filed: Jul. 17, 2001
(86) PCT No.: PCT/GB01/03221

(30) Foreign Application Priority Data
Jul. 17, 2000 (GB) 0017854036
Aug. 1, 2000 (GB) 0018857.3

(51) Int. Cl. C07D 211/20

(52) U.S. Cl. 546/236

(57) ABSTRACT

A process for preparing compound (E) from compound (A), with or without isolation of intermediate products, characterised by one or more of the following steps:

(1) reacting the sulphonate compound (B) with the substituted phenol in the presence of a phase transfer catalyst and a base,

(2) reacting compound (C) and the haloformate with addition of an HCl scavenging base,

(3) washing the reaction solution containing compound (D) with an aqueous acid selected from citric acid, phosphoric acid, acetic acid and formic acid,

(4) heating compound (D) with sodium hydroxide to remove the carbamate group.

Preferably the reaction(s) take place in toluene, providing an advantageous procedure for commercial production of paroxetine.

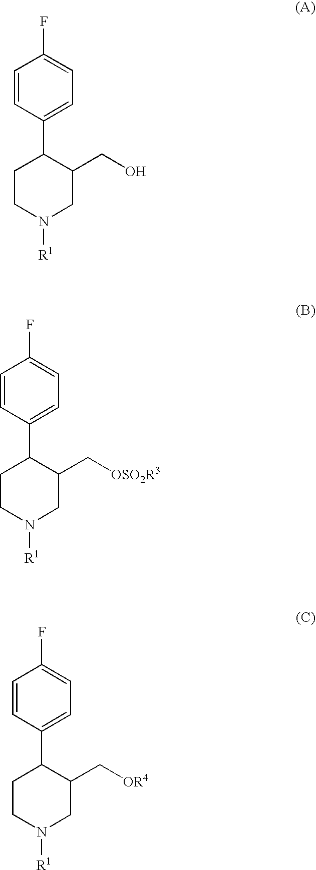

The present invention relates to new processes for preparing pharmaceutically active compounds and intermediates therefor.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxy-phenoxymethyl)-piperidine. This compound is used in therapy as the hydrochloride salt to treat inter alia depression, obsessive compulsive disorder (OCD). and panic.

Previously published processes to paroxetine utilise, as exemplified by Engelstoft and Hansen in Acta Chemica Scandinavica 1996: 50: 164-169 and in U.S. Pat. No. 4,007,196, as a key intermediate the carbinol (A)

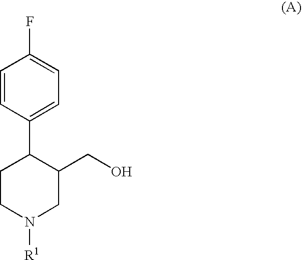

in which the piperidine nitrogen is protected by a group R¹, usually an alkyl (typically methyl) or aralkyl (such as benzyl) group. The N-substituted piperidine must be coupled with sesamol to make an N-substituted paroxetine analogue (C′)

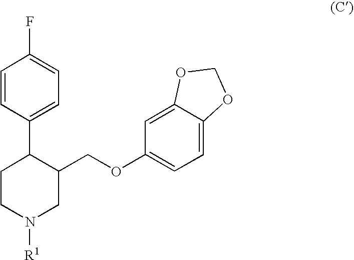

which is converted to paroxetine (compound (E′) below) by removal of the nitrogen protecting group

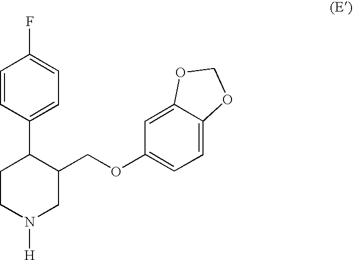

In the above references, coupling with the alcohol proceeds via a sulphonate ester intermediate (B)

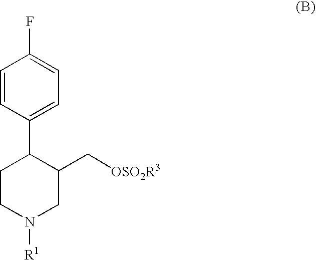

where R³ is typically a lower alkyl, aralkyl or aryl group, such as methyl, benzyl or phenyl.

In U.S. Pat. No. 3,912,743, Example 1, a solution of 3-hydroxymethyl-1-methyl-4-phenyl piperidine in pyridine is reacted with methanesulphonyl chloride. The pyridine is removed and the crude resultant sulphonate ester is treated with sodium methoxide and 4-methoxyphenol in methanol under reflux. In Example 5 of EP 0 152 273, 4-(4fluorophenyl)-3-hydroxymethyl-1-methyl pyridine is dissolved in toluene together with triethylamine and cooled. Benzenesulphonyl chloride is added to this mixture. The resultant solution of the benzenesulphonic ester is then mixed with sodium methoxide and 4-methoxyphenol in methyl isobutyl carbinol and heated.

In the above references, removal of the nitrogen protecting group takes place via a carbamate intermediate. The following scheme illustrates the synthesis:

R¹ is typically lower alkyl (such as methyl) or aralkyl (such as benzyl) group,

R² is typically lower alkyl (such as methyl), aryl (such as phenyl), aralkyl (such as benzyl), or alkenyl (such as vinyl),

R⁴ is a substituted phenyl group (especially 3,4-methylenedioxyphenyl)

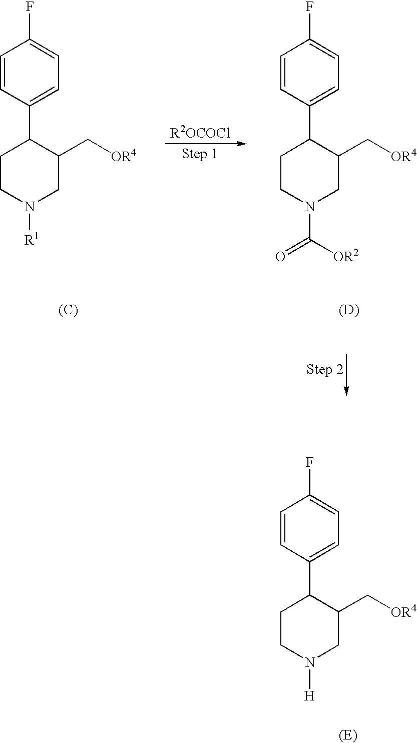

The prior art procedures for preparation of paroxetine isolate and purify the product of each step before proceeding to the next. This has been perceived as essential because of the physical similarities between the compounds of formula (C) and (E), such that any unreacted compound (C) passing through to the end of Step 2 above is impossible to remove using the conventional recrystallisation techniques. Comparatively, compounds (C) and (D) are quite different and so relatively easy to separate by crystallisation at the end of Step 1. There is also a general concern that impurities produced in Step 1, if not removed, will produce additional difficult to remove or at least previously unknown impurities in the process of Step 2.

In U.S. Pat. No. 4,007,196, Example 2, a solution of 4-fluorophenyl-3-(3′,4′-methylenedioxyphenoxymethyl)-1-methy piperidine in dichloromethane was treated with phenyl chloroformate in dichloromethane at 0-5° C. After leaving overnight, the solution was washed with 1M NaOH and then 1M HCl, dried and evaporated. The solid residue was suspended in benzene, filtered and evaporated. The evaporation residue was heated at reflux with KOH and 2-methoxyethanol for 4 hours and then evaporated. Water was added and the mixture extracted with benzene, dried, and evaporated to give the N-deprotected compound.

In Example 9 of EP 0 152 273, 4-(4′-fluorophenyl)-3-(4′-methoxy phenoxymethyl)-1-methyl piperidine was dissolved in toluene and treated at 0° C. with a solution of 1.9 equivalents of phenyl chloroformate in toluene over 30 minutes. The mixture was allowed to stand at room temperature for 20 hours. A further 1.9 equivalents of phenyl chloroformate were added and the mixture left for 72 hours. The solution was washed with 2N NaOH, then water, then 1N HCl and finally saturated aqueous NaCl. The organic phase was dried and concentrated to give an oil which was then crystallised as a white crystals from 96% ethanol. This intermediate was mixed with KOH and 2-methoxyethanol and stirred at 130-140° C. for 4 hours and partitioned between water and toluene. The organic phase was dried and evaporated to give the N-deprotected compound as an oil which was then converted to the acetate salt.

The present invention is based on the discovery of improvements in the above mentioned sulphonation, coupling and deprotection steps individually, and by combining steps, to provide reaction conditions which are more suitable for industrial scale production.

In one aspect the present invention provides a process for the preparation of a compound of structure (E):

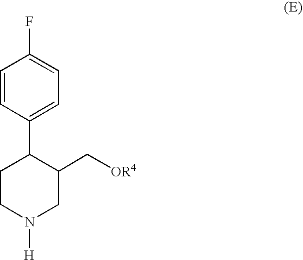

in which R⁴ is a substituted phenyl group (especially 3,4-methylenedioxyphenyl), which comprises

(a) providing a carbinol compound of structure (A)

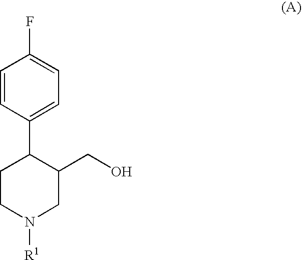

(b) reacting the carbinol with a sulphonyl chloride of structure R³SO₂Cl to prepare a sulphonate derivative of structure (B)

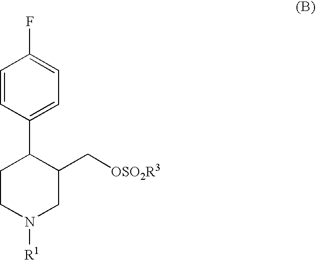

(c) reacting the sulphonate with a substituted phenol R⁴OH in the presence of a base to obtain a compound of structure (C)

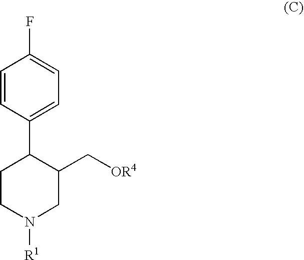

(d) reacting the compound (C) with a haloformate R²OCOCl to obtain a compound of structure (D)

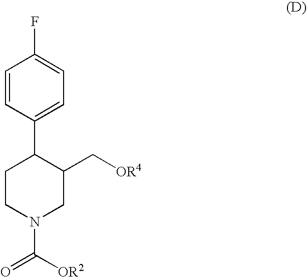

(e) treating compound (D) with a base to remove the carbamate group R²OCO— and obtain compound (E),

characterised in that the above reaction sequence is carried out starting with a solution of compound (A) and adding the reagents R³SO₂Cl, R⁴OH, R²OCOCl and bases, and appropriate reaction auxiliaries where necessary, to successive reaction solutions without isolation of the intermediate compounds (B), (C) and (D).

In the above process:

R¹ is suitably an alkyl, arylalkyl, allyl, arylalkyloxycarbonyl, acyl or alkynyl group in which the alkyl groups have 1-6 carbon atoms, preferably an alkyl (typically methyl) or aralkyl (such as benzyl) group,

R² is suitably an optionally substituted alkyl, aryl, allyl, alkenyl or arylalkyl group in which the alkyl groups have 1 to 6 carbon atoms, preferably a phenyl, methyl, ethyl, tertiary butyl, vinyl or benzyl group

R³ is suitably an alkyl, aralkyl, alkaryl or aryl group in which the alkyl groups have 1 to 6 carbon atoms, preferably a Ph, CF₃, CH₃, CH₂Ph, CH₂COPh, C₆H₄-4-MeO, C₆H₂-2,4,6-Me₃, C₆H₄-4-Me, CH₂Ph, or CH₂C₆H₄-4-Me group

R⁴ is a substituted phenyl group, suitably substituted by C₁-₄ alkyl, alkylthio, alkoxy, halogen, nitro, acylamino, methylsulfonyl, or preferably methylenedioxy,

Compound (E) may be isolated as the free base, or more preferably is converted to a salt with a pharmaceutically acceptable acid before isolation.

Most suitably the above reaction sequence is carried out in toluene, starting with a solution of compound (A) in toluene and adding reagents neat or as solutions in toluene or with additional toluene as make-up, as appropriate.

Typically the above reaction sequence may be carried out by adding a solution of the sulphonyl chloride, such as benzenesulphonyl chloride, in the reaction solvent, suitably toluene, to the solution of compound (A) with a base, preferably an amine such as dimethylethylamine, and allowing the reaction to take place at reduced temperature, suitably less than 20° C., for example between −10 and +5° C. Preferably the reaction solution is subjected to an aqueous wash before proceeding, most suitably using aqueous sodium hydroxide. After removal of the aqueous phase, the substituted phenol such as sesamol is added to the reaction solution together with a suitable base, such as aqueous sodium or potassium hydroxide. Suitably, if toluene is used as the reaction solvent then a phase transfer catalyst is added. The temperature is suitably maintained at about 60-100° C. during this reaction. Typically a further aqueous wash follows this reaction, and optionally drying of the reaction solution, for example by azeotropic distillation. Then the chloroformate, such as phenyl chloroformate, is added. Suitably the temperature is maintained between 50-100° C. Preferably the reaction solution is given a further aqueous wash, suitably with dilute sulphuric acid. Finally a base such as sodium or potassium hydroxide is added to remove the nitrogen-protecting carbamate, suitably by heating the reaction solution under reflux. The product may be recovered from the solution by various means, such as evaporation of solvent, precipitation by addition of a non-solvent or adding an acid such as hydrochloric acid to form a salt and crystallising the salt.

In a commercial process, for reasons of production logistics it may be desirable that compounds (C) and/or (D) are isolated before further reaction. In that case it may still be advantageous that steps (a) and (b) are carried out without isolation of compound (B). The preparation of compound (C) by carrying out steps (a), (b) and (c) in a common solvent, most suitably using toluene, without isolation of compound (B), forms another aspect of this invention.

In this aspect of the invention, step (d) may suitably be carried out by adding chloroformate to the reaction solution containing compound (C). Alternatively, compound (C) in solid form or in solution with the reaction solvent may be added to chloroformate in the reaction solvent.

As a further aspect of this invention we have devised improved procedures for each of the steps (b)-(e) in the above process for preparing compound (E) from compound (A). These procedures may be used effectively both when the process is carried out as a non-isolation process in a common base solvent as disclosed above, or when the steps are carried out sequentially with isolation of intermediate compounds before proceeding to the next step.

Accordingly the present invention also provides a process for preparing compound (E) from compound (A) by steps (a), (b), (c), (d) and (e) above, characterised by one or more of the following improvements:

(1) in step (c) reacting the sulphonate compound (B) with the substituted phenol in the presence of a phase transfer catalyst and a base,

(2) in step (d) reacting compound (C) with the haloformate and adding an HCl scavenging base,

(3) in step (d) washing the reaction solution containing compound (D) with an aqueous acid selected from citric acid, phosphoric acid, acetic acid and formic acid,

(4) in step (e) heating compound (D) with sodium hydroxide to remove the carbamate group.

The individual steps (1)-(4) also form separate aspects of this invention.

Accordingly the invention also provides:

A process for preparing compound (C) which comprises reacting a sulphonate compound (B) with a substituted phenol R⁴OH in the presence of a phase transfer catalyst and a base.

A process for preparing compound (D) which comprises reacting a compound (C) and a haloformate R²OCOCl and adding an HCl scavenging base.

A process for preparing compound (D) which comprises reacting a compound (C) with a haloformate R²OCOCl and washing the reaction solution containing compound (D) with an aqueous acid selected from citric acid, phosphoric acid, acetic acid and formic acid.

A process for preparing compound (E) which comprises heating a compound (D) with sodium hydroxide to remove the carbamate group.

Phase Transfer Catalyst

In operating improvement (1) above, typically a solution of compound (B) is provided in a reaction vessel, and appropriate amounts of the substituted phenol e.g. sesamol, the phase transfer catalyst e.g. tetra-n-octylammonium bromide, and base e.g. aqueous sodium hydroxide, and optionally additional solvent, are added to the vessel. The mixture is heated and stirred to effect the reaction.

A suitable phase transfer catalyst for this process is tetra-n-octylammonium bromide, which may be used effectively when the reaction is carried out in toluene, a solvent especially suitable for use in commercial production.

Another suitable phase transfer catalyst for this process is tetra-n-butylammonium bromide, which may be used effectively when the reaction is carried out in toluene and may advantageously be removed from the reaction solution after step (c) without loss of yield.

Other phase transfer catalysts that may be used for this reaction include tetra-n-dodecylammonium chloride, Aliquat/Adogen (R)464, tetra-n-butylammonium chloride, cetyltrimethylammonium bromide, and tetra-n-butylammonium fluoride hydrate.

The phase transfer catalysed reaction may also be carried in other solvents, such as benzene, xylene, mesitylene and other hydrocarbons.

However the reaction preferably takes place in toluene. In that case it is advantageous that compound (B) is also formed by a reaction in toluene, for example by addition of a sulphonyl chloride in toluene to a solution of compound (A) in toluene in the presence of a base, such as an amine. After suitable work-up, such as washing with aqueous base, for example 10% NaOH, the reaction solution containing compound (13) in toluene can be used directly for reaction with the phenol R⁴OH, without the need for intermediate isolation of compound (B).

In the formation of compound (B), the base is preferably an amine, such as triethylamine, trimethylamine, diethylmethylamine or dimethylethylamine. More preferably, the amine is dimethylethylamine.

Most suitably, a solution of benzenesulphonyl chloride in toluene is added dropwise to a solution of compound (B), especially trans-(−)-4-(4′-fluorophenyl)-3-hydroxymethyl-1-methylpiperidine (R¹=methyl), and dimethylethylamine in toluene, keeping the temperature of the reaction below 20° C. The reaction mixture is then preferably washed with aqueous sodium hydroxide solution. To the toluene solution is added a substituted phenol, such as sesamol, a phase transfer catalyst (preferably tetra-n-octylammonium bromide), suitably at 1-5 mol %, typically at 1-3 mol %, and aqueous (typically 30-50 w/w %) sodium hydroxide solution. The mixture is heated for reaction, typically at 70-100° C. The toluene solution is then separated, optionally washed with water, and concentrated. The product may be isolated for further reaction to form compound (D), or the reaction solution used directly in a non-isolation process as disclosed above.

We have found that the above process of this invention may be carried out with high yields relative to the uncatalysed procedure, but with the advantage of using a low cost catalyst that is active at low loadings, and with readily available materials such as toluene and sodium hydroxide.

HCl Scavenging Base

This aspect of the invention results from the finding that the reaction between the haloformate and the substituted phenol derivative (C) to prepare compound (D) does not always proceed to completion because any HCl present in the reaction mixture reacts with starting material to form its hydrochloride salt. This precipitates from solution and so does not contribute to the reaction product. As a result, we have found that a variable amount, typically from 5-12%, of the starting material may remain unreacted at the end of the reaction.

This aspect of the invention is based on the surprising discovery that HCl scavenging bases, especially hindered amine bases such as Hunig's base (ethyldiisopropylamine) can be used successfully to allow the reaction to be driven to completion. This gives higher conversions and yields in the production of compound (D).

Advantageously the base is added after commencement of the reaction between the chloroformate and compound (C), most suitably after the initial reaction has been completed, for example about 30 minutes after addition of the chloroformate. The hindered amine may be selected by reference to possessing sufficient basicity to scavenge HCl but being insufficiently nucleophilic to react with the chloroformate. We believe that previous attempts to use HCl scavenging bases have failed due to their being present at the commencement of the reaction, and resultant reaction with the chloroformate. Suitably the base is added in amount of 0.1 to 1 equivalents.

Acid Wash

Whether carried out as a non-isolation process or as a separate step, the reaction solution containing compound (D) resulting from the reaction of compound (C) with the haloformate is typically subjected to an aqueous wash as part of the work-up procedure. It has been proposed to wash with aqueous sulphuric acid which converts unreacted starting material to a sulphate or hydrogen sulphate, which separates as an oil. The oil must be removed, for example by Celite filtration, during work-up. Reducing the amount of unreacted starting material by the above-mentioned HCl-scavenging process will also have the effect of reducing the amount of oil to be removed during work-up.

We have now found that it is more advantageous to wash the reaction solution with an aqueous acid selected from citric acid, phosphoric acid, acetic acid and formic acid, to remove traces of unreacted compound (C) before further treatment of compound (D).

This aspect of the invention is based on the surprising finding that use of citric acid, phosphoric acid, acetic acid and formic acid in the aqueous wash converts the unreacted starting material to salts that are sufficiently soluble in the aqueous phase to avoid the formation of oil, and so which can be extracted in the wash liquid.

Preferably the selected acid is provided in the wash at 0.5 to 2M and washing is carried out with the wash solution at about 20-60° C. Preferred acids are citric and phosphoric, especially citric acid.

Deprotection

To complete the preparation of compound (E), compound (D) is treated with a base to remove the carbamate group. Preferably this takes place by adding the base to the reaction solution containing compound (D). However, if desired, compound (D) may be isolated by evaporation of solvent, and the evaporation residue treated with the base, or the isolated compound (D) may be taken up in fresh solvent before reaction.

For example in a typical procedure, potassium hydroxide may be added to a solution of compound (D), and the mixture is heated under reflux for several hours.

However we have found that it is surprisingly advantageous to carry out the final deprotecting step in the preparation of compound (E), in particular paroxetine, by using sodium hydroxide as the base to remove the carbamate group.

For the reaction to operate efficiently, it is preferable that it takes place in the presence of water. The optimal amount of water depends on the reaction concentration and the amount of NaOH used.

Control of the amount of water present is difficult to achieve when using potassium hydroxide in an industrial scale process because commercial potassium hydroxide flake contains varying amounts of water.

Because of the greater reliability of the specification of commercial sodium hydroxide, we have found that we can carry out the deprotection step with significantly reduced amounts of base and solvent when compared to the use of potassium hydroxide. For example, a process which required 7.4 equivalents of potassium hydroxide and 15 volumes of toluene can be carried out using 4 equivalents of sodium hydroxide in 7.5 volumes of toluene, thus effectively doubling the throughput of the reaction vessel. Under these conditions the optimal amount of water for the reaction is provided by use of 20-25% water (w/w) relative to the sodium hydroxide. The water and sodium hydroxide (as pearl) may be added separately.

Suitably the final reaction solution is washed with water and optionally aqueous sodium hydroxide, before recovering compound (E) from the solution, for example by evaporation of solvent or crystallisation. In the case of paroxetine the deprotection step is preferably carried out in toluene. After washing, a cosolvent such as propan-2-ol or industrial methylated spirits (IMS) may be added to the toluene solution, and hydrochloric acid added to allow crystallisation of paroxetine as the hydrochloride salt, optionally after seeding.

The compound of structure (E) may isolated from the final solution as the free base for purification or for further reaction to create active compounds or salts as in U.S. Pat. No. 3,912,743 and U.S. Pat. No. 4,007,196 and the references cited above. Preferably the above procedures are used to prepare paroxetine, in which case R⁴ is a residue of sesamol.

Paroxetine is the (−)-trans isomer of 4-(4′-fluorophenyl)-3-(3′,4′-methylenedioxyphenoxymethyl)-piperidine. Following the procedure of EP-0 152 273, optical resolution may be carried out prior to coupling with sesamol. Alternatively, resolution may be carried out at other stages, such as after deprotection of the piperidine nitrogen.

The present invention includes within its scope the compound paroxetine and its pharmaceutically acceptable salts, particularly paroxetine hydrochloride, especially as an anhydrate or the hemihydrate, and paroxetine methanesulphonate, when obtained via any aspect of this invention, and any novel intermediates resulting from the described procedures.

Paroxetine free base may be converted to paroxetine methanesulphonate by treatment with methanesulphonic acid or a labile derivative thereof, for example a soluble salt such as ammonium methanesulphonate. Paroxetine hydrochloride may be prepared by treatment of paroxetine free base with a source of hydrogen chloride, for example gaseous hydrogen chloride, or a solution thereof, or aqueous hydrochloric acid.

Paroxetine and its salts obtained using this invention may be formulated for therapy in the dosage forms described in EP-A-0223403 or WO96/24595, either as solid formulations or as solutions for oral or parenteral use.

Therapeutic uses of paroxetine, especially paroxetine hydrochloride or methanesulphonate, obtained using this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Pharmaceutical compositions using active compounds prepared in accordance with this invention are usually adapted for oral administration, but formulations for dissolution for parental administration are also within the scope of this invention.

The composition is usually presented as a unit dose composition containing from 1 to 200 mg of active ingredient calculated on a free base basis, more usually from 5 to 100 mg, for example 10 to 50 mg such as 10, 12.5, 15, 20, 25, 30 or 40 mg by a human patient. Most preferably unit doses contain 20 mg of active ingredient calculated on a free base basis. Such a composition is normally taken from 1 to 6 times daily, for example 2, 3 or 4 times daily so that the total amount of active agent administered is within the range 5 to 400 mg of active ingredient calculated on a free base basis. Most preferably the unit dose is taken once a day.

Preferred unit dosage forms include tablets or capsules, including formulations adapted for controlled or delayed release.

The compositions of this invention may be formulated by conventional methods of admixture such as blending, filling and compressing. Suitable carriers for use in this invention include a diluent, a binder, a disintegrant, a colouring agent, a flavouring agent and/or preservative. These agents may be utilised in conventional manner, for example in a manner similar to that already used for marketed anti-depressant agents.

Accordingly, the present invention also provides:

a pharmaceutical composition for treatment or prophylaxis of one or more of the Disorders comprising paroxetine or a pharmaceutically acceptable salt such as the mesylate or hydrochloride obtained using the process of this invention and a pharmaceutically acceptable carrier;

the use of paroxetine or a pharmaceutically acceptable salt such as the mesylate or hydrochloride obtained using the process of this invention to manufacture a medicament for the treatment or prophylaxis of one or more of the Disorders; and

a method of treating the Disorders which comprises administering an effective or prophylactic amount of paroxetine or a pharmaceutically acceptable salt such as the mesylate or hydrochloride obtained using the process of this invention to a person suffering from one or more of the Disorders.

This invention is illustrated by the following Examples.

### EXAMPLE 1

Toluene (250 ml) and trans-(−)-4-(4′-fluorophenyl)-3-hydroxymethyl-1-methylpiperidine (63 g) were charged to a 1 litre jacketed vessel at 20° C. with an agitation rate of 300 r.p.m. The temperature was lowered to 5° C., then dimethylethylamine (42.8 ml) was charged to the vessel and the solution temperature was lowered to 0° C. A solution of benzenesulphonyl chloride (43.3 ml) in toluene (32 ml) was added to the vessel over 120 minutes, maintaining the solution temperature between +2 and −2° C. On completion of the addition, the solution was stirred at +2 to −2° C. for 2 hours. A solution of sodium hydroxide in water (10% w/w, 66.3 g) was added to the reactor over 15 minutes. The mixture was warmed to 20° C., stirred for 15 minutes then left to settle. After 15 minutes, the aqueous phase was removed. To the toluene solution was added tetra-n-octylammonium bromide (4.63 g), sesamol (43.25 g), toluene (6.3 ml) and aqueous sodium hydroxide (100.7 g in 201.7 ml of water). The reaction was warmed to 70° C. and stirred with an agitator speed set to 300 r.p.m. for 8 hours. The reaction was diluted with water (283 ml) stirred for 10 minutes then left to settle. After 15 minutes, the aqueous phase was removed. The reaction was cooled to 30° C. and washed with water (2×63 ml). The toluene was then removed by distillation under reduced pressure. To the resulting solid was added propan-2-ol (315 ml) and the resulting suspension was heated to 80° C. to dissolve all the solid. The propan-2-ol was removed by distillation under reduced pressure to leave a solid that was left under reduced pressure at 50° C. for 1 hour after the distillation was complete. To the solid was added propan-2-ol (95 ml) and the suspension was heated to reflux and stirred until all the solid dissolved. The reaction was cooled to 64° C. and seeded with trans-(−)-4-(4′-fluorophenyl)-3-(3″,4″-methylenedioxyphenoxymethyl)-1-methylpiperidine (20 mg). The reaction was then cooled slowly to 20° C. and water (378 ml) was added over 1 hour. The reaction was cooled to 15° C. and stirred for 1 hour. The suspension was filtered and the solid washed with water (150 ml) and dried in a vacuum oven at 40° C. overnight. The yield of trans-(−)-4-(4′-fluorophenyl)-3-(3″,4″-methylenedioxyphenoxymethyl)-1-methylpiperidine was 92.5 g, (95.5%) with a purity of 95.4%.

### EXAMPLE 2

Toluene (400 ml) and trans-(−)-4-(4′-fluorophenyl)-3-hydroxymethyl-1-methylpiperidine (100 g) were charged to a 1 litre jacketed vessel at 20° C. with an agitation rate of 300 r.p.m. The temperature was lowered to 5° C., then dimethylethylamine (68.4 ml) was charged to the vessel and the solution temperature was lowered to 0° C. A solution of benzenesulphonyl chloride (69.1 ml) in toluene (50 ml) was added to the vessel over 65 minutes, maintaining the solution temperature between +2 and −2° C. On completion of the addition, the solution was stirred at +2 to −2° C. for 2 hours. A solution of sodium hydroxide in water (10% w/w, 25 ml of 40% w/w NaOH made up to 100 ml with water) was added to the reactor over 15 minutes. The mixture was warmed to 20° C., stirred for 15 minutes then left to settle. After 15 minutes, the aqueous phase was removed. To the toluene solution was added tetra-n-octylammonium bromide (2.49 g), sesamol (69.07 g), toluene (10 ml) and aqueous sodium hydroxide (228 g of 47% w/w solution). The reaction was warmed to 75° C. and stirred with an agitator speed set to 360 r.p.m. for 3 hours. The reaction was diluted with water (500 ml) stirred for 15 minutes then left to settle. After 15 minutes, the aqueous phase was removed. The reaction was cooled to 40° C. and washed with water (100 ml). The toluene was then removed by distillation under reduced pressure. To the resulting solid was added propan-2-ol (500 ml) and the resulting suspension was heated to 80° C. to dissolve all the solid. The propan-2-ol was removed by distillation under reduced pressure to leave a solid that was left under reduced pressure at 50° C. for 1 hour after the distillation was complete. To the solid was added propan-2-ol (150 ml) and the suspension was heated to reflux and stirred until all the solid dissolved. The reaction was cooled to 64° C. and seeded with trans-(−)-4-(4′-fluorophenyl)-3-(3″,4″-methylenedioxyphenoxymethyl)-1-methylpiperidine (20 mg). The reaction was then cooled slowly to 20° C. and water (600 ml) was added over 1 hour. The reaction was cooled to 15° C. and stirred for 1 hour. The suspension was filtered and the solid washed with water (240 ml) and dried in a vacuum oven at 40° C. overnight. The yield of trans-(−)-4-(4′-fluorophenyl)-3-(3″,4″-methylenedioxyphenoxymethyl)-1methylpiperidine was 147.7 g, (96.0%) with a purity of 96.8%.

### EXAMPLE 3

Trans-(−)-4-(4′-fluorophenyl)-3-(3″,4″-methylenedioxyphenoxymethyl)-1-methylpiperidine (20 g, 58.2 mmol) was slurried in toluene (120 ml) and dried by the azeotropic distillation of 20 ml solvent. The slurry was then cooled to 65° C. and phenylchloroformate (8.0 ml, 63.7 mmol) added over ca. 40 min, whilst maintaining the reaction temperature at 60-65° C. When the addition was complete, the reaction mixture was stirred at 60-65° C. for 1 hour.

The reaction mixture was washed at 60° C. with 2M citric acid (2×60 ml), followed by water (2×30 ml), then evaporated to dryness to give an oil. This was dissolved in hot propan-2-ol (110 ml) and again evaporated to dryness to give a white solid. The residue was redissolved in hot propan-2-ol (144 ml) and allowed to cool. The resulting solid was collected by filtration to give 19.9 g (76%) of trans-(−)-4-(4′-fluorophenyl)-3-(3″,4″-methylenedioxyphenoxymethyl)-1-phenoxycarbonylpiperidine.

### EXAMPLE 4

Trans-(−)-4-(4′-fluorophenyl)-3-(3″,4″-methylenedioxyphenoxymethyl)-1-methylpiperidine (22.5 g, 0.066 mol) and toluene (130 ml) were charged to a 500 ml 3-necked, round-bottomed flask. The mixture was dried by distillation of toluene (20 ml) and cooled to 60-65° C. Phenylchloroformate (9 ml, 0.072 mol, 1.1 eq.) was added over 45 minutes, then di-iso-propylethylamine (2.3 ml, 0.013 mol, 0.2 eq.) added and the mixture left to stir at 60-65° C. for 1 hour. After cooling to 20° C. the mixture was washed with 10% sulphuric acid (2×30 ml) and water (2×38 ml). The mixture was treated with celite (0.63 g), filtered and the solvent evaporated. Propan-2-ol (125 ml) was added and the solvent again evaporated. Fresh propan-2-ol (160 ml) was added and the mixture heated to give a solution. This was cooled to 0-5° C., stirred for 2 hours and filtered. The product was washed with propan-2-ol (20 ml) and dried at 40° C. under vacuum. The yield of trans-(−)-4-(4′-fluorophenyl)-3-(3″,4″-methylenedioxyphenoxymethyl)-1-phenoxycarbonylpiperidine was 27.05 g (91.8%) with a purity of 98.3%.

### EXAMPLE 5

Trans-(−)-4-(4′-fluorophenyl)-3-(3″,4″-methylenedioxyphenoxymethyl)-1-phenoxycarbonylpiperidine (75 g) and toluene (560 ml) were charged to a 1 L jacketed vessel and the agitator set to a speed of 400 rpm. Water (8.6 ml) and sodium hydroxide pearl (29.53 g) were added and the mixture was heated at reflux for 60 minutes then cooled to 75° C. Water (170 ml) was added and the mixture stirred for 5 minutes at 70-75° C. then settled for 5 minutes. The lower aqueous layer was removed then a second portion of water (170 ml) was added and the mixture stirred for 5 minutes at 70-75° C. then settled for 5 minutes. After removing the lower aqueous layer the toluene phase was cooled to 40° C. and industrial methylated spirit (16.5 ml) was added. The mixture was further cooled to 18° C. when seeds of trans-(−)-4-(4′-fluorophenyl)-3-(3″,4″-methylenedioxyphenoxymethyl)-piperidine hydrochloride hemihydrate (27 mg) were added followed immediately by concentrated hydrochloric acid (23 ml). The mixture was then recooled to 20-22° C. and stirred for 1 hour. The toluene slurry was filtered and the resulting cake was washed with fresh toluene (75 ml). The product was dried in a vacuum oven (55° C.) overnight. The yield of trans-(−)-4-(4′-fluorophenyl)-3-(3″,4″-methylenedioxyphenoxymethyl)-piperidine hydrochloride hemihydrate was 61.44 g (98.2%) with a purity of 98.4%.

### EXAMPLE 6

Toluene (375 ml) and trans-(−)-4-(4′-fluorophenyl)3-hydroxymethyl-1-methylpiperidine (63 g) were charged to a 1 litre jacketed vessel at 20° C. with an agitation rate of 300 r.p.m. The temperature was lowered to 5° C., then dimethylethylamine (46 ml) was charged to the vessel and the solution temperature was lowered to 0° C. A solution of benzenesulphonyl chloride (43 ml) in toluene (51 ml) was added to the vessel over 1 hour 45 minutes, maintaining the solution temperature between 0 and +2° C. On completion of the addition, the solution was stirred at 1° C. for 3 hours. A solution of sodium hydroxide in water (6 g in 300 ml of water) was added to the reactor over 15 minutes. The mixture was warmed to 20° C. stirred for 15 minutes then left to settle. After 15 minutes, the aqueous phase was removed. To the toluene solution was added tetra-n-octylammonium bromide (4.62 g), sesamol (43 g), toluene (300 ml) and aqueous sodium hydroxide (203.4 g in 405 ml of water). The reaction was warmed to 70° C. and stirred with an agitator speed set to 300 r.p.m. for 18 hours. The reaction was cooled to room temperature, diluted with water (600 ml), stirred for 15 minutes then left to settle. After 15 minutes, the aqueous phase was removed. Toluene (330 ml) was then removed from the reaction by distillation. The reaction solution was then transferred to a clean 1 litre jacketed vessel using toluene (75 ml) as a wash. A further portion of toluene (80 ml) was removed by distillation. The mixture was cooled to 60° C. and phenylchloroformate (42 ml) was added over 45 minutes. N,N-di-iso-propylethylamine (10 ml) was then added and the mixture left to stir at 60° C. for 1 hour. The reaction was cooled to 20° C. and washed with 2M citric acid solution (2×380 ml) and water (2×160 ml). The solution was transferred to a Buchi flask and the solvent was evaporated in vacuo. Propan-2-ol (540 ml) was charged to the flask and also evaporated in vacuo. A further portion of propan-2-ol (700 ml) was added to the residue, which was heated until all the solid dissolved. The heating bath was removed and the mixture allowed to cool. After cooling to 0-5° C. for 1 hour, the white solid was filtered, washed with propan-2-ol (2×100 ml) and dried overnight at 40° C. under vacuum. The yield of trans-(−)-4-(4′-fluorophenyl)-3-(3″,4″-methylenedioxyphenoxymethyl)-1-phenoxycarbonylpiperidine was 102.8 g (81%) with a purity of 98%.

### EXAMPLE 7

Toluene (380 ml) and trans-(−)-4-(4′-fluorophenyl)-3-hydroxymethyl-1-methylpiperidine (63 g) were charged to a 1 litre jacketed vessel at 20° C. with an agitation rate of 300 r.p.m. The temperature was lowered to 5° C., then dimethylethylamine (43.8 ml) was charged to the vessel and the solution temperature was lowered to 0° C. A solution of benzenesulphonyl chloride (42.6 ml) in toluene (51 ml) was added to the vessel over 2 hours 45 minutes, maintaining the solution temperature between +2 and −2° C. On completion of the addition, the solution was stirred at 1° C. for 3 hours. A solution of sodium hydroxide in water (6 g in 300 ml of water) was added to the reactor over 15 minutes. The mixture was warmed to 20° C., stirred for 15 minutes then left to settle. After 15 minutes, the aqueous phase was removed. To the toluene solution was added tetra-n-octylammonium bromide (4.62 g), sesamol (42.9 g), toluene (25 ml) and aqueous sodium hydroxide (101.7 g in 204 ml water). The reaction was warmed to 70° C. and stirred with an agitator speed set to 300 r.p.m. for 18 hours. The reaction was cooled to room temperature, diluted with water (283 ml), stirred for 15 minutes then left to settle. After 15 minutes, the aqueous phase was removed. To the reaction was added toluene (300 ml) and toluene (300 ml) was then removed from the reaction by distillation. The reaction solution was then transferred to a clean 1 litre jacketed vessel using toluene (100 ml) as a wash. A further 88 ml of toluene was removed by distillation. The mixture was cooled to 60° C. and phenylchloroformate (39 ml) was added over 45 minutes. N,N-di-iso-propylethylamine (10 ml) was then added and the mixture left to stir at 60° C. for 1 hour. The reaction was then cooled to 20° C. and washed with 2M citric acid (2×380 ml) and water (2×166 ml). Toluene (468 ml) was then added, and removed (100 ml) by distillation. The reaction was cooled to 50° C. and the agitator set to a speed of 400 rpm. Water (19.5 ml) and sodium hydroxide pellet (45.3 g) were added and the mixture was heated at reflux for 75 minutes then cooled to 80° C. Water (285 ml) was added and the mixture stirred for 5 minutes at 80° C. then settled for 60 minutes. The lower aqueous layer was removed then a second portion of water (285 ml) was added and the mixture stirred for 5 minutes at 80° C. then settled for 5 minutes. After removing the lower aqueous layer, the toluene phase was cooled to 40° C. and industrial methylated spirit (28 ml) was added. The mixture was further cooled to 18° C., seeds of with trans-(−)-4-(4′-fluorophenyl)-3-(3″,4″-methylenedioxyphenoxymethyl)-piperidine hydrochloride hemihydrate (0.086 g) were added, followed immediately by concentrated hydrochloric acid (39 ml). The mixture was then cooled to 20-22° C. and stirred for 1 hour. The toluene slurry was filtered and the resulting cake was washed with fresh toluene (80 ml). The product was dried in a vacuum oven at 55° C. overnight. Yield of trans-(−)-4-(4′-fluorophenyl)-3-(3″,4″-methylenedioxyphenoxymethyl)-piperidine hydrochloride hemihydrate was 93.66 g (88.6%) with a purity of 97.5%.

### EXAMPLE 8

(−) Trans-4-(4′-fluorophenyl)-3-(3″,4″-methylenedioxyphenoxymethyl)-N-methylpiperidine (67.5 g) and toluene (340 ml) were charged to a 500 ml conical flask and stirred to give a hazy solution. The solution was filtered through celite and washed through with fresh toluene (70 ml). The filtered toluene solution was transferred to a 3 neck 500 ml round bottom flask equipped with a Dean and Stark distillation apparatus. The toluene solution was heated to reflux and dried by azeotropic distillation of toluene at atmospheric pressure collecting 70 ml of distillate. After completion of the distillation the toluene solution was cooled to 60-65° C. then phenylchloroformate (26 ml) was added dropwise over 45 minutes, using a syringe pump, maintaining the temperature at 60-65° C. On completion of the addition the mixture was stirred for 30 minutes at 60-65° C. then ethyldiisopropylamine (3.3 ml) was added in one portion and the mixture stirred for another 30 minutes. The mixture was then transferred to a 1 L jacketed vessel and the temperature adjusted to 40-45° C. 2M Citric acid (170 ml) was added and the mixture stirred at 40-45° C. for 5 minutes and settled for 5 minutes. The lower aqueous layer was removed and a second portion of 2M citric acid (170 ml) added. The mixture was stirred for 5 minutes at 40-45° C., settled for 5 minutes and the lower layer was removed. Water (70 ml) was then added and the mixture stirred at 40-45° C. for 5 minutes and settled for 10 minutes. The lower aqueous layer was removed and a second portion of water (70 ml) added. The mixture was stirred for 5 minutes at 40-45° C., settled for 10 minutes and the lower layer was removed. The toluene solution was concentrated in vacuo keeping the internal temperature around 40-50° C. to give a thick oil. IPA (300 ml) was added to the oil and heated to around 70° C. until a solution formed. The IPA solution was concentrated in vacuo keeping the internal temperature around 40-50° C. to give a solid. IPA (405 ml) was added to the solid and heated to reflux and stirred until complete dissolution. The IPA solution was cooled to 60-65° C., stirred for 30 minutes, then cooled to 20-22° C. and stirred for 1 hour. The IPA slurry was filtered and the resulting cake was washed with fresh IPA (70 ml). The product was dried in a vacuum oven (55° C.) overnight.

Yield of (−) trans-4-(4′-fluorophenyl)-3-(3″,4″-methylenedioxyphenoxymethyl)-N-phenoxycarbonylpiperidine: 79.9 g, 90.4% by weight. Purity 98.1%.

### EXAMPLE 9

(−) Trans-4-(4′-fluorophenyl)-3-(3″,4″-methylenedioxyphenoxymethyl)-N-phenoxycarbonylpiperidine (22 g) and toluene (165 ml) were charged to a 250 ml jacketed vessel and the agitator set to a speed of 400 rpm. Water (2.5 ml) and sodium hydroxide pearl (8.7 g) were added and the mixture was heated at 95° C. for 60 minutes then cooled to 75° C. Water (50 ml) was added and the mixture stirred for 5 minutes at 70-75° C. then settled for 5 minutes. The lower aqueous layer was removed then a second portion of water (50 ml) was added and the mixture stirred for 5 minutes at 70-75° C. then settled for 5 minutes. After removing the lower aqueous layer the toluene phase was cooled to 40° C. and industrial methylated spirit (4.8 ml) was added. The mixture was further cooled to 18° C. when seeds (˜10 mg) were added followed immediately by concentrated hydrochloric acid (6.8 ml). The mixture was then recooled to 20-22° C. and stirred for 1 hour. The toluene slurry was filtered and the resulting cake was washed with fresh toluene (22 ml). The product was dried in a vacuum oven (55° C.) overnight.

Yield of (−) trans-4-(4′-fluorophenyl)-3-(3″,4″-methylenedioxyphenoxymethyl)-piperidine hydrochloride hemihydrate: 97.7% by weight. Purity 97.7%.

### EXAMPLE 10

(−) Trans-4(4′-fluorophenyl)-3-(3″,4″-methylenedioxyphenoxymethyl)-N-methylpiperidine (67.5 g) and toluene (340 ml) were charged to a 500 ml conical flask and stirred to give a hazy solution. The solution was filtered through celite and washed through with fresh toluene (70 ml). The filtered toluene solution was transferred to a 3 neck 500 ml round bottom flask equipped with a Dean and Stark distillation apparatus. The toluene solution was heated to reflux and dried by azeotropic distillation of toluene at atmospheric pressure collecting 70 ml of distillate. After completion of the distillation the toluene solution was cooled to 60-65° C. then phenylchloroformate (25.5 ml) was added dropwise over 45 minutes, using a syringe pump, maintaining the temperature at 60-65° C. On completion of the addition the mixture was stirred for 30 minutes at 60-65° C. then ethyldiisopropylamine (3.2 ml) was added in one portion and the mixture stirred for another 30 minutes. The mixture was then transferred to a 1 L jacketed vessel and the temperature adjusted to 40-45° C. 2M Citric acid (170 ml) was added and the mixture stirred at 40-45° C. for 5 minutes and settled for 5 minutes. The lower aqueous layer was removed and a second portion of 2M citric acid (170 ml) added. The mixture was stirred for 5 minutes at 40-45° C., settled for 5 minutes and the lower layer was removed. Water (70 ml ) was then added and the mixture stirred at 40-45° C. for 5 minutes and settled for 10 minutes. The lower aqueous layer was removed and a second portion of water (70 ml) added. The mixture was stirred for 5 minutes at 40-45° C., settled for 10 minutes and the lower layer was removed. The toluene solution was diluted with more toluene (440 ml), heated to reflux and dried by azeotropic distillation of toluene at atmospheric pressure collecting 135 ml of distillate. The toluene solution was cooled to 75° C. and the agitator was adjusted to a speed of 400 rpm. Water (9.6 ml) and sodium hydroxide pellet (33.12 g) were added and the mixture was heated at reflux for 60 minutes then cooled to 75° C. Water (170 ml) was added and the mixture stirred for 5 minutes at 70-75° C. then settled for 5 minutes. The lower aqueous layer was removed then a second portion of water (170 ml) was added and the mixture stirred for 5 minutes at 70-75° C. then settled for 5 minutes. After removing the lower aqueous layer the toluene phase was cooled to 40° C. and industrial methylated spirit (19 ml) was added. The mixture was further cooled to 18° C. when seeds (40 mg) were added followed immediately by concentrated hydrochloric acid (25 ml). The mixture was then recooled to 20-22° C. and stirred for 1 hour. The toluene slurry was filtered and the resulting cake was washed with fresh toluene (70 ml). The product was dried in a vacuum oven (55° C.) overnight.

Yield of (−) trans-4-(4′-fluorophenyl)-3-(3″,4″-methylenedioxyphenoxymethyl)-piperidine hydrochloride hemihydrate: 88.9% by weight. Purity 97.4%.

### EXAMPLE 11

Toluene (80 ml) and trans-(−)-4-(4′-fluorophenyl)-3-hydroxymethyl-1-methylpiperidine (20 g) were charged to a 250 ml jacketed vessel at 20° C. The temperature was lowered to 10° C., then dimethylethylamine (13.6 ml) was charged to the vessel and the solution temperature was lowered to 0° C. A solution of benzenesulphonyl chloride (13.7 ml) in toluene (10 ml) was added to the vessel over 60 minutes, maintaining the solution temperature between +2 and −2° C. On completion of the addition, the solution was stirred at +2 to −2° C. for 1 hour. A solution of sodium hydroxide in water (10% w/w, 20 ml) was added to the reactor over 5 minutes. The mixture was warmed to 20° C., stirred for 15 minutes then left to settle. After 15 minutes, the aqueous phase was removed. To the toluene solution was added tetra-n-butylammonium bromide (1.45 g), sesamol (13.74 g), and aqueous sodium hydroxide (32 ml of 47% w/w solution). The reaction was warmed to 75° C. and stirred for 2.5 hours. The reaction was diluted with water (100 ml) stirred for 15 minutes then left to settle. After 15 minutes, the aqueous phase was removed. The reaction was cooled to 55° C. and washed with water (20 ml). The toluene was then removed by distillation under reduced pressure. To the resulting solid was added propan-2-ol (100 ml) and the resulting suspension was heated to 80° C. to dissolve all the solid. The propan-2-ol was removed by distillation under reduced pressure to leave a solid. To the solid was added propan-2-ol (30 ml) and the suspension was heated to reflux and stirred until all the solid dissolved. The reaction was cooled to 60° C. and seeded with trans-(−)-4-(4′-fluorophenyl)-3-(3″,4″-methylenedioxyphenoxymethyl)-1-methylpiperidine (5 mg). The reaction was then cooled slowly to 20° C. and water (120 ml) was added over 30 minutes. The reaction was cooled to 15° C. and stirred for 1 hour. The suspension was filtered and the solid washed with water (48 ml) and dried in a vacuum oven at 55° C. overnight. The yield of trans-(−)-4-(4′-fluorophenyl)-3-(3″,4″-methylenedioxyphenoxymethyl)-1-methylpiperidine was 28.9 g, (94.0%) with a purity of 98.3%.

### EXAMPLE 12

Toluene (480 ml) was charged to a 1L jacketed vessel, dried by the azeotropic distillation of 80 ml, then cooled to 65° C. Phenyl chloroformate (35.5 ml) was charged to the vessel. The solution was maintained at 60 to 65° C. and (−) trans-4-(4′-fluorophenyl)-3-(3″,4″-methylenedioxyphenoxymethyl-N-methylpiperidine (80 g) was added in 10 equal portions over 45 minutes, then washed in with toluene (2 ml). On completion of the addition the mixture was stirred for 30 minutes at 60-65° C. then di-iso-propylethylamine (3.95 ml) was added in one portion and the mixture stirred for another 30 minutes. The mixture was then cooled to 40-45° C. 2M Citric acid (200 ml) was added and the mixture stirred at 40-45° C. for 5 minutes and settled for 5 minutes. The lower aqueous layer was removed and a second portion of 2M citric acid (200 ml) added. The mixture was stirred for 5 minutes at 40-45° C., settled for 5 minutes and the lower layer was removed. Water (80 ml) was then added and the mixture stirred at 40-45° C. for 5 minutes and settled for 10 minutes. The lower aqueous layer was removed and a second portion of water (80 ml) added. The mixture was stirred for 5 minutes at 40-45° C., settled for 10 minutes and the lower layer was removed. The toluene solution was concentrated in vacuo keeping the internal temperature around 40-50° C. to give a thick oil. IPA (360 ml) was added to the oil and heated to around 70° C. until a solution formed. The IPA solution was concentrated in vacuo keeping the internal temperature around 40-50° C. to give a solid. IPA (480 ml) was added to the solid and heated to reflux and stirred until complete dissolution. The IPA solution was cooled to about 65° C., then stirred at 60-70° C. for 30 minutes. The resultant slurry was then cooled to 20-22° C. and stirred for 1 hour. The IPA slurry was filtered and the resulting cake was washed with fresh IPA (80 ml). The product was dried in a vacuum oven (55° C.) overnight.

Yield of (−) trans-4-(4′-fluorophenyl)-3-(3″,4″-methylenedioxyphenoxymethyl)-N-phenoxycarbonylpiperidine was 99.0 g, (94.5%) with a purity of 98.2%.

### EXAMPLE 13

Toluene (350 ml) and (−) trans-4-(4′-fluorophenyl)-3-(3″,4″-methylenedioxyphenoxymethyl)-N-methylpiperidine (100 g) were charged to a 500 ml jacketed vessel. The solution was dried by the azeotropic distillation of 50 ml of toluene, then cooled to about 65° C. Toluene (150 ml) and phenyl chloroformate (44.4 ml) were charged to a 1 L jacketed vessel and heated to 60-65° C. The toluene solution of (−) trans-4-(4′-fluorophenyl)-3-(3″,4″-methylenedioxyphenoxymethyl)-N-methylpiperidine was then added to the toluene solution of phenyl chloroformate over 45 minutes. When the addition was complete, the transfer lines were washed through with toluene (50 ml). The mixture was then stirred for 30 minutes at 60-65° C. then di-iso-propylethylamine (4.9 ml) was added in one portion and the mixture stirred for another 30 minutes. The mixture was then cooled to 40-45° C. 2M Citric acid (250 ml) was added and the mixture stirred at 40-45° C. for 5 minutes and settled for 5 minutes. The lower aqueous layer was removed. Water (100 ml) was then added and the mixture stirred at 40-45° C. for 5 minutes and settled for 10 minutes. The lower aqueous layer was removed and a second portion of water (100 ml) added. The mixture was stirred for 5 minutes at 40-45° C., settled for 10 minutes and the lower layer was removed. The toluene solution was concentrated in vacuo keeping the internal temperature around 40-50° C. to give a thick oil. IPA (400 ml) was added to the oil and heated to around 70° C. until a solution formed. The IPA solution was concentrated in vacuo keeping the internal temperature around 40-50° C. to give a solid. IPA (600 ml) was added to the solid and heated to reflux and stirred until complete dissolution. The IPA solution was cooled to about 65° C., then stirred at 60-70° C. for 30 minutes. The resultant slurry was then cooled to 20-22° C. and stirred for 1 hour. The IPA slurry was filtered and the resulting cake was washed with fresh IPA. (100 ml). The product was dried in a vacuum oven (55° C.) overnight.

Yield of (−) trans-4-(4′-fluorophenyl)-3-(3″,4″-methylenedioxyphenoxymethyl)-N-phenoxycarbonylpiperidine was 124.2 g, (94.9%) with a purity of 98.6%.

1. A process for the preparation of a compound of structure (E): 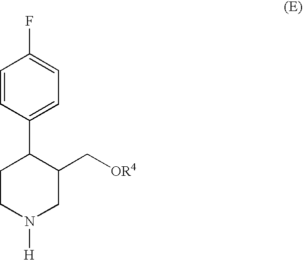
in which R⁴ is a substituted phenyl group (especially 3,4-methylenedioxyphenyl), which comprises
(a) providing a carbinol compound of structure (A) 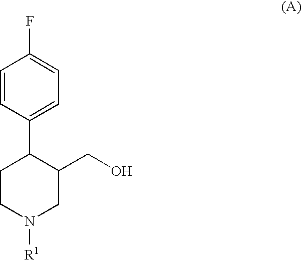
(b) reacting the carbinol with a sulphonyl chloride of structure R³SO₂Cl to prepare a sulphonate derivative of structure (B) 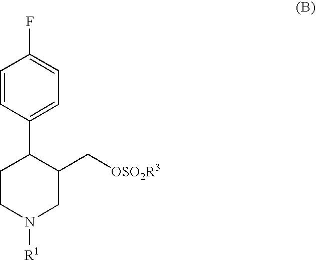
(c) reacting the sulphonate with a substituted phenol R⁴OH in the presence of a base to obtain a compound of structure (C) 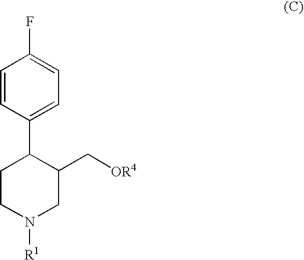
(d) reacting the compound (C) with a haloformate R²OCOCl to obtain a compound of structure (D) 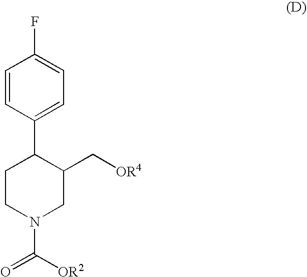
(e) treating compound (D) with a base to remove the carbamate group R²OCO— and obtain compound (E),
characterised in that the above reaction sequence is carried out starting with a solution of compound (A) and adding the reagents R³SO₂Cl, R⁴OH, R²OCOCl and bases to successive reaction solutions without isolation of the intermediate compounds (B), (C) and (D).

2. A process for preparing compound (E) from compound (A) by steps (a), (b), (c) and (d) of claim 1, with or without isolation of intermediate products, characterised by one or more of the following improvements:
(1) in step (c) reacting the sulphonate compound (B) with the substituted phenol in the presence of a phase transfer catalyst and a base,
(2) in step (d) reacting compound (C) with the haloformate and adding an HCl scavenging base,
(3) in step (d) washing the reaction solution containing compound (D) with an aqueous acid selected from citric acid, phosphoric acid, acetic acid and formic acid,
(4) in step (e) heating compound (D) with sodium hydroxide to remove the carbamate group.

3. A process for preparing compound (C) as defined in claim 1 which comprises reacting a sulphonate compound (B) as defined in claim 1 with a substituted phenol R⁴OH in the presence of a phase transfer catalyst and a base.

4. A process for preparing compound (D) as defined in claim 1 which comprises reacting a compound (C) as defined in claim 1 and a haloformate R²OCOCl and adding an HCl scavenging base.

5. A process for preparing compound (D) as defined in claim 1 which comprises reacting a compound (C) as defined in claim 1 with a haloformate R²OCOCl and washing the reaction solution containing compound (D) with an aqueous acid selected from citric acid, phosphoric acid, acetic acid and formic acid.

6. A process for preparing compound (E) as defined in claim 1 which comprises heating a compound (D) as defined in claim 1 with sodium hydroxide to remove the carbamate group.

7. A process according to any preceding claim in which the substituted phenol R⁴OH is sesamol.

8. A process according to any preceding claim in which R³ is phenyl.

9. A process according to any preceding claim in which R² is phenyl.

10. A process according to any preceding claim in which R¹ is methyl.

11. A process according to any one of claims 2-10, in which the phase transfer catalyst is tetra-n-octylammonium bromide or tetra-n-butylammonium bromide.

12. A process according to any one of claims 2-11, in which the HCl-scavenging base is Hunig's base.

13. A process according to any preceding claim in which the reaction takes place in toluene.

14. Paroxetine, or a pharmaceutically acceptable salt thereof, obtainable by or using a process as claimed in any preceding claim.

15. A method of treating the Disorders which comprises administering an effective or prophylactic amount of paroxetine or a pharmaceutically acceptable salt such as the mesylate or hydrochloride obtainable using the process of any one of claims 1 to 13 to a person suffering from one or more of the Disorders.
